(19) United States
(12) Patent Application Publication
(10) Pub. No.: US 2002/0138091 A1
(43) Pub. Date: Sep. 26, 2002

(54) Micro-invasive nucleotomy device and method

(75) Inventor: D. Russell Pflueger, San Juan Capistrano, CA (US)

Correspondence Address: Frank J. Uxa, Stout, Uxa, Buyan & Mullins, LLP, Suite 300, 4 Venture, Irvine, CA, 92618, US

(73) Assignee: DevonRex, Inc., San Juan Capistrano, CA (US)

(21) Appl. No.: 10/093,774

(22) Filed: Mar. 8, 2002

Related U.S. Application Data
(60) Provisional application No. 60/278,128, filed on Mar. 23, 2001.
(60) Provisional application No. 60/281,848, filed on Apr. 5, 2001.
(60) Provisional application No. 60/305,178, filed on Jul. 13, 2001.
(60) Provisional application No. 60/322,909, filed on Sep. 17, 2001.
(60) Provisional application No. 60/342,436, filed on Dec. 21, 2001.

(51) Int. Cl. A61B 17/32

(52) U.S. Cl. 606/170

(57) ABSTRACT

An apparatus and method for removing intervertebral disc material, particularly nucleus pulposus, from a patient is provided. The apparatus generally includes a handpiece and a tissue removal mechanism operatively coupled thereto. The tissue removal mechanism includes a cannula adapted to be inserted into an intervertebral disc and having an open distal tip and an outer diameter of less than about 5 mm, or less than about 2 mm. The mechanism further includes a rotatable element having a distal portion with projections or threading, for example, having a substantially helical configuration. The distal portion of the rotatable element may extend beyond the open distal tip of the cannula. The apparatus is designed to urge or draw nucleus material into the cannula upon rotation of the rotatable element and preferably without the need for supplemental sources of suction or aspiration. The invention further provides a method for monitoring intervertebral disc pressure before, during and/or after a treatment procedure.

### RELATED APPLICATION

This application claims the benefit of U.S. provisional applications Serial No. 60/278,128, filed Mar. 23, 2001 and Serial No. 60/281,848, filed Apr. 5, 2001 and Serial No. 60/305,178, filed Jul. 13, 2001 and Serial No. 60/322,909, filed Sep. 17, 2001 and Serial No. 60/342,436, filed Dec. 21, 2001, the disclosure of each of which is incorporated in its entirety herein by reference.

### FIELD OF THE INVENTION

The present invention relates generally to medical devices and methods, and, more particularly, to medical devices and methods for accessing intervertebral discs and removing tissue therefrom, for example, for accessing and removing tissue from a disc nucleus.

### BACKGROUND OF THE INVENTION

The medical industry is constantly evolving through the adaptation of improved pharmaceutical, biotechnology, and medical device products and procedures. Techniques and technologies are being developed to treat internal areas of the body through less invasive means.

Recently, devices have been developed to explore and therapeutically impact areas inside the spinal canal. These devices are primarily designed to reduce the amount of pain that chronic pain patients are experiencing due to abnormal conditions existing in and around the spinal cord and intervertebral disc. Devices currently used to treat these areas include: spinal injections of anesthetics and anti-inflammatories, RF and cryo neuroablation, epiduroscopes, infusion catheters, spinal stimulation devices, micro endoscopic discectomy instruments, and the like.

The spinal column includes, among other structures, the bony vertebrae which surround the spinal cord, and the intervertebral discs. In a healthy spine, the discs maintain separation between the vertebrae, promote fluid circulation throughout the spine, and provide a cushioning effect between the bony vertebral structures.

Due to the elastic nature of an intervertebral disc, the disc is subject to injury if the disc becomes overstressed, for example, by trauma to the spine, excess body weight, improper mechanical movements and the like. Intervertebral disc injuries and other abnormalities result in serious back pain and physical disability and are often chronic and difficult to treat. Such abnormalities include, but are not limited to, localized tears or fissures in the disc annulus, localized disc herniations with contained or escaped nuclear extrusions, and circumferential bulging discs. Discs also experience degeneration over time which can accelerate these problems.

Disc fissures may result from structural degeneration of fibrous components of the disc annulus (annulus fibrosis). More specifically, fibrous components of the annulus become separated in particular areas, creating a fissure within the annulus. Sometimes the fissure is accompanied by extrusion of material from the disc nucleus (nucleus pulposus), into the fissure. Biochemicals may escape from the disc and irritate surrounding structures. These disc fissures are known to be extremely painful. The fissure may also be associated with herniation of that portion of the annulus wall.

With a contained disc herniation, the nucleus pulposus may work its way partly through the annulus. The outward protrusion of fibrous and nuclear material can press upon the spinal nerves or irritate other body structures.

Another common disc problem occurs when the entire disc bulges circumferentially about the annulus rather than in specific, isolated locations. This may occur for example, when over time, the disc weakens, bulges, and takes on a “roll” shape. The joint may become unstable and one vertebrae may eventually settle on top of another. This problem typically continues to escalate as the body ages, and accounts for shortened stature in old age. Osteophytes may form on the outer surface of the disc and further encroach upon the spinal canal and nerve foramina. This condition is called spondylosis.

Traditional non-surgical treatments of disc degeneration and abnormalities include bed rest, pain and muscle relaxant medication, physical therapy or steroid injection. Such therapies are directed primarily at pain relief and delaying further disc degeneration. In many cases, non-surgical approaches fail and surgical methods of treatment may be applied. Spinal fusion methods are aimed at causing the vertebrae above and below the injured disc to grow solidly together forming a single piece of bone. This procedure is carried out with or without discectomy (surgical removal of the disc). Another procedure, endoscopic discectomy, involves removing tissue from the disc percutaneously in order to reduce the volume of the disc, thereby reducing impingement of the surface of the disc on nearby nerves.

Endoscopic Discectomy is an outpatient surgical procedure to remove herniated disc material. Using local anesthesia with the help of x-ray video image for guidance, an endoscopic probe is inserted between the vertebrae and into the herniated disc space through the skin of the back. Surgical attachments (cutters, lasers, and the like) are then sent down the hollow center of the probe to remove a portion of the offending disc. Sometimes, the surgical attachments can be used to push the bulging disc back into place and for the removal of disc fragments and small bone spurs. This form of discectomy utilizes the same tools used for knee surgery but maneuvers the instruments above the spine. The surgeon introduces the endoscope through a large approximately 10 mm or greater, incision into the skin above the spine, then locates the nerve and disc using direct visualization. This surgery can be done through the abdomen for anterior discectomy as well. These procedures are performed under direct endoscopic visualization which increases the incisional space requirement and may require a hemi-laminectomy (surgical removal of part of the lamina).

Summers U.S. Pat. No. 5,383,884, discloses a spinal disc surgical instrument including a rotating cutting shaft including a lateral cutting port for progressively shaving away discrete portions of a herniated disc until the herniated disc is completely removed. The instrument also includes a non-rotating idler shaft for evacuating severed tissue from the surgical site.

There still exists a need for an apparatus for safely and effectively treating an intervertebral disc, for example, by removing material or tissue, for example, nucleus pulposus from within the disc.

### SUMMARY OF THE INVENTION

New apparatus and methods for removing material, for example, tissue and/or other material from a human or animal spinal column, for example but not exclusively, intervertebral discs, have been discovered. The present invention provides apparatus, for example, micro-invasive apparatus, to remove material, for example, tissue or other material from a target area of a human or animal intervertebral disc to provide one or more benefits, such as diagnostic benefits, therapeutic benefits and the like.

The apparatus of the invention are useful for removing unwanted, diseased, or even healthy bodily materials, for example, nucleus pulposus within an intervertebral disc, for medical treatment and/or therapeutic purposes. Advantageously, the present invention is suitable for use in many surgical settings and is suitable for performing various material removal procedures using methodologies, for example, in terms of methods of introducing the apparatus into the body and removing the apparatus from the body, which are substantially analogous to conventional surgical techniques. Necessary or desirable adaptations of the apparatus of the present invention for specific medical treatment, e.g., diagnostic, and therapeutic purposes will be readily appreciated by those of skill in the art.

Accordingly, apparatus for removing material from an intervertebral disc area of a human or animal spinal column are provided. In one broad aspect, the apparatus comprise a handpiece and a tissue removal element connected or coupled thereto. The tissue removal element includes a cannula, for example, a substantially rigid or flexible cannula, and a rotational element disposed at least partially in the cannula. The rotational element is structured to be operatively coupled to a source of rotational energy, for example, a motor. The cannula includes an open distal tip structured to be placed in a nucleus of an intervertebral disc of a body, and preferably a proximal end portion structured to be coupled, for example, removably coupled, to the handpiece. The rotational element is structured to at least assist in drawing material from an intervertebral disc into the cannula, for example, into the open distal tip of the cannula, in response to, for example, as a result of, rotation of the rotational element relative to the cannula.

In one embodiment, the rotational element is structured to at least assist in drawing material from an intervertebral disc into the cannula. For example, the rotational element and the cannula cooperatively engage to form or create a source of suction effective in drawing, preferably sufficient to draw, the material from the intervertebral disc into the cannula in response to rotation of the rotational element relative to the cannula. Advantageously, the cannula, in particular the interior hollow space formed or defined by the cannula, and the rotational element are sized and positioned, relative to each other, to create a source of suction or pumping action in response to the rotational element rotating relative to the cannula.

Without wishing to limit the invention to any particular theory of operation, it is believed that this functioning of the cannula/rotational element combination is at least somewhat analogous to the functioning of a pump, for example, a pump based on the principles of the “Archimedes' screw”, causing the material to be drawn or pulled or pumped into the open distal tip of the cannula and through the cannula in being removed from the intervertebral disc of the human/animal body.

Preferably, the suction/pumping action created or formed by the cannula/rotational element combination is itself sufficient and effective to draw material into the cannula so that no other, for example, no additional or supplemental, source of suction, aspiration or pumping action is employed, needed or required to effectively remove material from the intervertebral disc, for example, the nucleus pulposus, in accordance with the present invention.

In one embodiment of the invention, the rotational element includes a shaft and one or more outwardly extending projections, for example, threads, preferably having a substantially helical configuration. Advantageously, the rotational element includes a distal portion with such projections or threads. The proximal portion of the rotational element may or may not include such projections or threads. In a very useful embodiment, the proximal portion is substantially free of such projections or threads.

The distal portion of the rotational element, in a useful embodiment, extends beyond the open distal tip or inlet of the cannula, for example, by a distance in a range of about 0.02 inches to about 1 inch beyond the open distal tip of the cannula. In one embodiment of the invention, the rotational element extends beyond the open distal tip by a distance equal to at least about one-half of the spacing between adjacent projections or threads. The rotational element distal portion may extend a distance equal to more than about one spacing, for example, about two spacings or more, between adjacent projections or threads beyond the open distal tip of the cannula. More specifically, the rotational element advantageously further includes an elongated shaft having a proximal portion which is substantially smooth to allow sufficient annular space between the shaft and cannula for removal of material.

The cannula may be of any suitable size. However, in order to obtain the reduced invasiveness or micro-invasiveness benefits of the present invention, it is preferred that the cannula size be no larger than about 5 mm, and more preferably about 2 mm or smaller.

It has unexpectedly been found that the present apparatus including such small size cannulas provide for reduced, or even micro, invasive procedures (which reduce surgical trauma and promote healing) and are effective in removing materials from a spinal column to achieve therapeutic benefits, for example, therapeutic removal of spinal disc nucleus tissue to effect decompression of a herniated disc.

In one embodiment of the invention, the open distal tip of the cannula is angled or is beveled with respect to a longitudinal axis of the cannula. Alternatively, the open distal tip is substantially perpendicular with respect to the longitudinal axis of the cannula.

The present apparatus advantageously includes a tissue collection chamber in communication, for example, fluid communication, with the cannula and structured to collect and contain material, for example, nucleus pulposus, passed through the cannula. The collection chamber preferably is structured to facilitate quantification and/or other analysis of the material removed from the intervertebral disc. In one particularly useful embodiment, the collection chamber comprises a substantially transparent conical section removably engaged to a housing of the handpiece and preferably circumscribing a portion, for example, the proximal portion of the shaft of the rotational element.

The cannula and/or the rotational element, preferably both, advantageously are structured to be manually deformable, for example, to enable the physician to alter the normal configuration, for example, the normal substantially straight configuration, thereof to create a curved configuration if desired, for example, to better access the disc or specific locations within the disc.

Advantageously, the apparatus of the present invention may be structured as a self-contained hand-held device which requires no external wiring or conduits for operation. The apparatus conveniently requires only single hand operation and is sufficiently lightweight so as to be easily maneuverable by a physician.

In another broad aspect of the present invention, methods of removing material from a spinal column of a human or an animal are provided. Such methods comprise placing into a spinal column, for example, a intervertebral disc, a cannula having an open distal tip and a rotational element disposed at least partially in the cannula, and rotating the rotational element relative to the cannula, thereby at least assisting in drawing a material from the intervertebral disc into the open distal tip of the cannula. The method preferably further comprises passing the material from the body through the cannula. Apparatus in accordance with the present invention described herein can be advantageously employed in accordance with the present methods.

The cannulas used in accordance with the present methods preferably have outer diameters of about 5 mm or less, for example, 2 mm or less.

The placing step of the present methods preferably includes percutaneously introducing the cannula into the nucleus of the disc, and positioning the open distal tip of the cannula in close proximity to the material, for example, nucleus pulposus, to be removed. The cannula and rotational element preferably are sized and positioned relative to each other so that the rotating step is effective in drawing the material from the disc of a human or an animal into the open distal tip of the cannula. Preferably, the material from the disc is removed without applying additional suction or aspiration to the open distal tip of the cannula.

In one very useful embodiment, the rotating of the rotational element relative to the cannula is effective to draw the nucleus into the cannula as a substantially single continuous piece. Thus, although some shearing and/or cutting of the nucleus to be removed may occur in accordance with the present invention, for example, so that the removed material is compatible with the space within the cannula through which the material is to be moved proximally, the present apparatus and methods preferably are not dependent or based on cutting or chopping the material to be removed into small discrete segments.

The present methods preferably further comprise collecting the intervertebral material removed and/or observing and/or otherwise testing the material removed.

The present invention further provides methods for treating and/or monitoring the status of an intervertebral disc by measuring and/or monitoring pressure in the intervertebral disc independent of, before, during and/or after a disc treatment procedure, for example, in order to achieve a safe and successful patient outcome. The methods preferably are used in conjunction with surgical procedures, wherein at least a portion of a disc nucleus is removed, or otherwise modified in order to benefit the spinal column, for example, to effect decompression of an intervertebral disc, for example, a herniated disc.

It is known that an intervertebral disc nucleus has an intrinsic pressure. In the event the disc pressure becomes elevated, for example, due to injury or trauma, the disc itself may bulge, or nucleus material from the center of the disc may extrude through fissures in the annulus and impinge on nearby nerves, causing severe pain and physical disability.

As described elsewhere herein, various surgical techniques are known which are directed at reducing the extent to which an intervertebral disc presses against nearby nerve structures. The present invention provides an effective method for determining an initial disc pressure prior to such a surgical technique and a post surgery disc pressure, for example, a pressure within a desired range.

Also included within the scope of the present invention are methods for monitoring the intrinsic pressure in the disc nucleus during a surgical procedure, for example a surgical procedure directed at reducing disc size or disc pressure. Such a medical procedure may utilize aspiration alone, or in conjunction with cutting or ablation to reduce the volume of nucleus material within the disc. The use of enzymes suitable for dissolving the gelatinous nucleus material may be employed as part of the surgical procedure.

In one aspect, methods in accordance with the present invention generally comprise the step of measuring the intrinsic pressure within an intervertebral disc nucleus before, during and/or after medical treatment of the disc. The step of monitoring may be performed intermittently, periodically, or on a substantially continuous real-time basis. The methods of the present invention allow a physician to utilize the pressure information obtained from the disc in diagnosing a problem, determining potential or actual effectiveness of a treatment, and/or determining the degree of treatment necessary to achieve a desired result.

Incorporated herein by this specific reference are the entire disclosures of U.S. patent applications for Micro-invasive Tissue Removal Device, having Ser. No. (attorney docket no. D-3034), filed on even date herewith, and commonly assigned herewith, and U.S. patent application for Micro-invasive Breast Biopsy Device, having Ser. No. (attorney docket no. D-3026), filed on even date herewith, and commonly assigned herewith.

Each and every feature described herein, and each and every combination of two or more of such features, is included within the scope of the present invention provided that the features included in such a combination are not mutually inconsistent.

The present invention and the objects and advantages thereof will be more clearly understood and appreciated with respect to the following Detailed Description, when considered in conjunction with the accompanying Drawings.

### BRIEF DESCRIPTION OF THE DRAWINGS

The present invention will be more clearly understood and appreciated by the following detailed description and accompanying drawings of which:

FIG. 1 is a side view of the apparatus of the present invention, including a handpiece and a rotational element connected thereto;

FIG. 2 is a cross-sectional view of the apparatus of FIG. 1;

FIG. 3 is a view of a distal end of the rotational element.

FIG. 4 shows a curved distal end of a cannula in accordance with an embodiment of the invention.

FIGS. 5 and 6 show alternative distal ends of the cannula and rotational element in accordance with the present invention.

FIG. 7 is a diagram depicting a method in accordance with the present invention.

### DETAILED DESCRIPTION

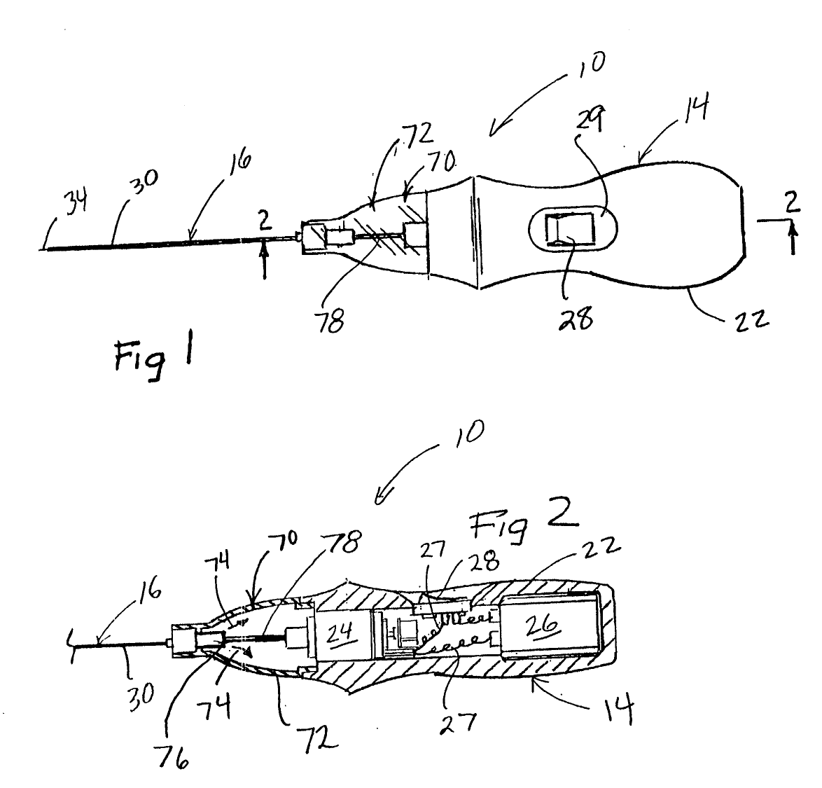

Turning now to FIGS. 1 and 2, a micro-invasive nucleotomy apparatus in accordance with the present invention is shown generally at 10. The apparatus 10 generally comprises a handpiece 14 and a tissue removal mechanism 16 to be described in detail hereinafter.

The handpiece 14 is preferably sized and contoured to fit comfortably within a palm of a surgeon, and includes for example a molded plastic housing 22. As shown in FIG. 2, the housing 22 of the handpiece 14 encloses a small motor 24 and a power supply, for example a 9 volt battery 26 for driving the tissue removal mechanism 16. Suitable electrical connectors 27 are provided. For convenient, one handed operation, an ON/OFF switch 28 is preferably provided on a recessed, lateral portion 29 of the housing 22.

Turning now as well to FIG. 3, the tissue removal mechanism 16 generally includes a cannula 30 and a rotatable element 34 disposed therein. As shown most clearly in FIG. 3, the cannula 30 includes a distal portion 40 defining an inlet 42 for receiving tissue drawn from a target area within a patient. The inlet 42 is defined, for example, by flat, distal edge 44 of the cannula 30. The distal edge 44, in the embodiment shown in FIG. 3, lies along a plane that is substantially perpendicular with respect to the longitudinal axis of the cannula 30. During operation of the apparatus 10, as will be described in greater detail hereinafter, tissue and/or other material is drawn, suctioned or pumped, through the inlet 42 and into a cylindrical bore 46 defined between the cannula 30 and a shaft 50 of the rotatable element 34.

In a preferred embodiment of the invention, such as shown in FIGS. 1-3, the tissue removal mechanism 16 is structured to draw tissue into the cannula 30 by a pumping action produced by rotation of the rotatable element 34, preferably without the use of supplemental aspiration or other means for drawing tissue into the threaded distal portion 52 or cannula 30. In other words, the rotational element 34 and the cannula 30 are designed to cooperatively engage to form a source of suction that is, in itself, sufficient to draw the tissue material into the cannula 30. Advantageously, the present invention 10 has been found to be safe and highly efficient for removing soft tissues from the body less invasively, without being connected to external sources of aspiration or other external machines and devices. In the preferred embodiment of the invention, the rotational element 34 includes a distal portion 52 which extends beyond the open distal tip (defined by edge 44) of the cannula 30. More preferably, the distal portion 52 extends a length of about 0.066 inches beyond the cannula distal edge 44. A blunt, rounded tip 53 of the rotational element 34 is preferably provided.

As shown, the rotational element 34 includes one or more outwardly extending projections, for example threads such as helical threading 56 shown, disposed about at least a portion of the shaft 50, for urging tissue into the bore 46. Preferably, outer radial edge 58 of the threading 56, or other projection, is disposed closely proximate an inner wall 62 of the cannula. As shown, the distal end 52 of the rotational element 34 extends at least one-half thread turn beyond the cannula inlet 42. This structure allows spinal tissue material to prolapse between the outer, distal-most threading turns, and be pulled into the inlet without necessarily being discretely cut or severed by the threading 56. The present invention is designed such that upon insertion of the open distal tip of the cannula 30 into the target region of the spine, disc nucleus material or other material will prolapse into and at least partially fill the open spaces between the projections or threading 56. Rotation of the rotational element 34, for example at about 12,000 RPM, causes the tissue material to be pulled in a proximal direction proximally into the bore 46, for example, as a continuous piece or strand of material.

Although the threading 56 is only shown as a single thread located on the distal portion 52 of the rotational element 34, it is to be appreciated that in some embodiments of the invention, the threading 56 may involve multiple threads, and/or may be disposed on more proximally located portions of the rotatable element shaft 50. Furthermore, although only about 4.5 turns of threading 56 are shown, it is to be appreciated that in some embodiments of the present invention, fewer or more than 4.5 turns of threading 56 may be provided. It is also contemplated by the present invention that rather than continuous threading 56, the shaft 50 may be provided with discontinuous threading. It is contemplated that with appropriate modifications and the like, these and other structures may be provided which would operate in a manner similar to the pumping action provided by the structure shown.

Preferably, the cannula 30 is structured to be insertable into an intervertebral disc, for example by means of a pre-inserted rigid stylet, and has an outer diameter of less than about 5 mm, for example, an outer diameter of about 2 mm or less. It is contemplated and considered within the scope of the invention that the cannula can be made to be percutaneously insertable into a disc without first introducing a stylet therein. That is, the cannula itself may be made of a suitable material and structure to be sufficiently rigid and strong to effectively puncture the skin, underlying fatty tissue, muscle and disc annulus in order to directly access the disc nucleus. However, the present invention is oftentimes used in conjunction with a stylet, as the back muscles overlying and connected to the spinal column are particularly dense and strong and therefore may be difficult to penetrate without the use of a sharp stylet.

The cannula 30 is made of any suitable, medical grade material or materials, but is preferably somewhat rigid and bendable or manually deformable.

Advantageously, as will be appreciated by those of skill in the art, the apparatus 10 of the present invention is micro-invasive or minimally invasive to the patient. For example, the cannula 30 can be introduced into the target area of the spinal column by means of a conventional, rigid stylet (not shown) disposed through the cannula 30 (detached from the handpiece 14). The cannula/stylet are introduced percutaneously through the skin, underlying muscle/fatty tissue and into the target area, for example through the fibrous annulus of an intervertebral disc, such that the inlet 42 is positioned within or closely adjacent the nucleus pulposus. The stylet is then removed and the cannula 30 is left in place. The rotational element 34, attached to the handpiece 14, is then introduced into the cannula 30. Preferably, in order to minimize invasiveness of the procedure, this procedure is facilitated through the use of fluoroscopy and x-ray imaging techniques as known in the art, which do not require endoscopic or direct viewing of the target tissue.

Advantageously, unlike prior art surgical tissue removal devices, the action of the tissue removal mechanism 16 urges tissue such as nucleus pulposus into the cannula 30 in many instances a substantially continuous cohesive segment rather than in relatively smaller, distinct portions of the tissue. Generally, the cannula 30 and rotational element 34 are structured to cooperatively function in a manner that will form a source of suction within the cannula 30 when the rotational element 34 is rotated while the cannula inlet 42 is disposed within the target tissue. It has been found that the level of suction so created is sufficient to gently and effectively draw soft tissue, for example gelatinous, viscous, or any suitable spinal column tissue that can be drawn by the action of the present invention into the cannula without need for any other, for example, supplemental, source of suction applied to the inlet 42. For example, the suction formed or created is sufficient to pull or soft tissues into the open tip without causing damage to other structures such as the inner wall of a disc annulus.

The tissue removal mechanism 16 can be left to remain in substantially the same position within the target area during the tissue removal procedure, or alternatively may be advanced or withdrawn during the procedure, for example in a direction along the longitudinal axis of the cannula in order to facilitate tissue removal.

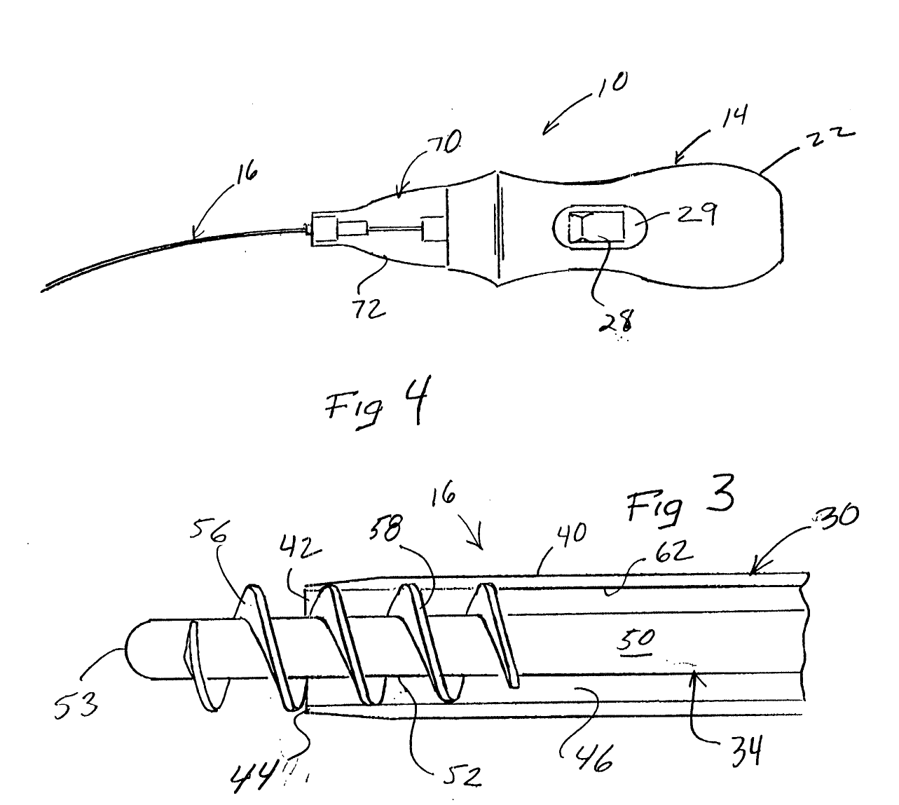

FIG. 4 shows another advantageous feature of the present invention. The tissue removal mechanism 16 may be structured to be deformed, for example, manually deformed, into a curve shape such as shown. The flexibility and deformability of the tissue removal mechanism 16 allows custom shaping or curving of the apparatus 10 for further facilitating access to tissue.

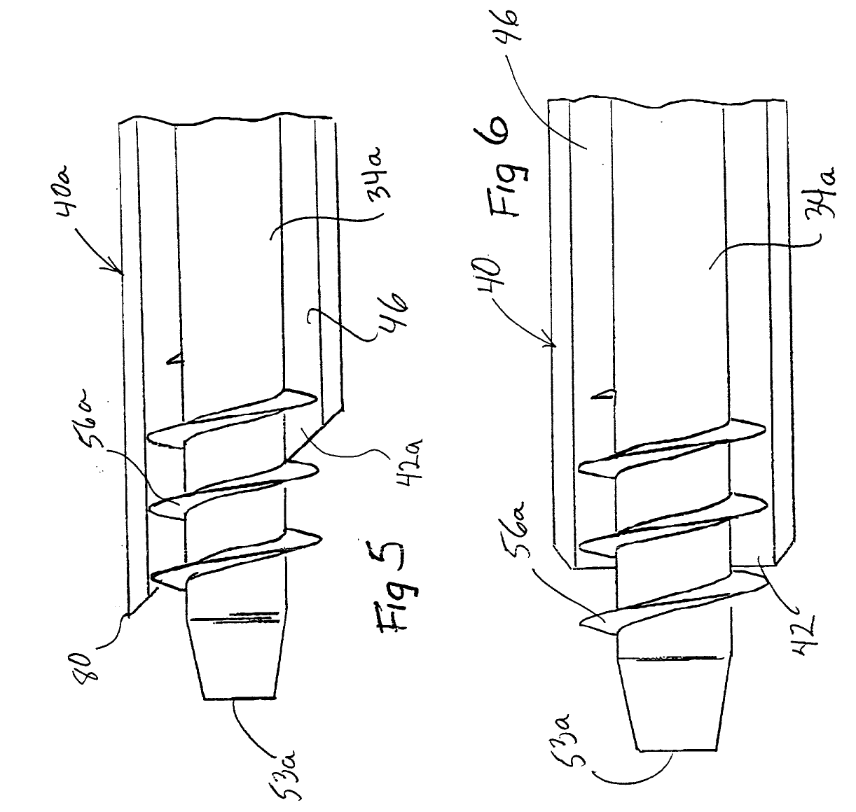

FIG. 5 shows an alternative cannula distal portion 40a, which is beveled, includes sharp distal tip 80, and a relatively wider inlet 42a than inlet 42. Also shown is a narrower threading 56a (relative to threading 56 of FIG. 3) on rotational element 34a. It is contemplated that in some embodiments of the present invention, a beveled cannula may be provided (such as in FIG. 5) and the rotational element may be somewhat recessed within the cannula, in that it does not extend further than a distal-most tip 80 thereof. Thus, it is contemplated that as long as at least a portion of threading is exposed to tissue through the angled inlet, the tissue will tend to prolapse between the threads and effectively be pulled into the inlet 42a and removed upon rotation of the rotatable element 34a.

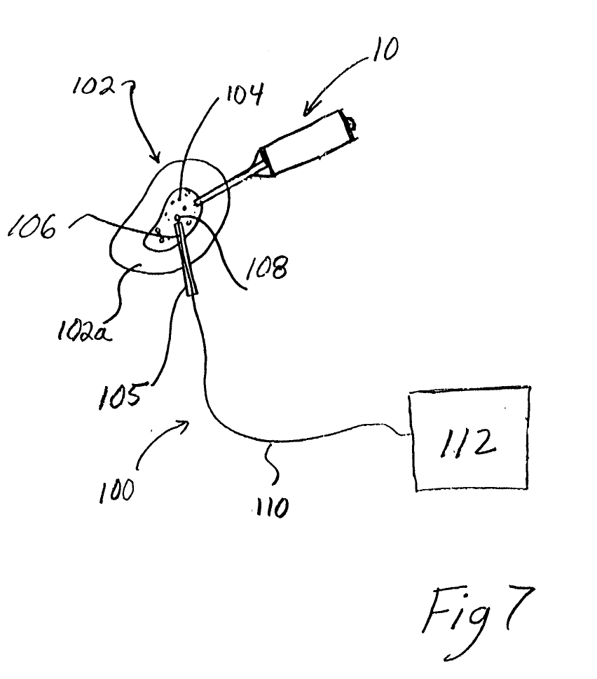

FIG. 6 shows a cannula distal portion 40 similar to that shown in FIG. 3. However the rotational element 34a is similar to that shown in FIG. 5, having narrow helical threading 56a, and a flat tip 53a rather than the rounded tip 53 shown in FIG. 3.

As shown in FIGS. 1, 2 and 4, the apparatus 10 may further comprise a collection chamber 70, for example, defined by a subhousing 72 removably engaged to the housing 22. More specifically, the collection chamber 72 is in fluid communication with a proximal portion 76 of the cannula 30. For example, the collection chamber 70 is adapted to collect, temporarily contain, and allow analysis of tissue, for example during and/or after the tissue removal procedure.

Generally, the collection chamber 70 is structured to contain material that is drawn from the surgical site. The removed material enters the collection chamber 70 as shown by arrows 74 in FIG. 2. The collection chamber 70 is preferably adapted to allow observation of the tissue material during the procedure. For example, the subhousing 72 may be transparent. In addition, the collection chamber 70 is preferably structured to allow quantification or measurement of the tissue, for example, the subhousing 72 may be provided with suitable indices (not shown) showing milliliters (ml) of material collected therein. As shown, a proximal portion 78 of the rotatable element 34 is circumscribed by the collection chamber 70.

It is further contemplated that in many applications of the present invention, the cannula 30 may alternatively or additionally be used as a passageway for introducing disc replacement material, medication, and/or other agents into the target region before or after the tissue removal, if desirable.

It can be appreciated that the present apparatus is less invasive in comparison to other percutaneous tissue removal devices in the art. Despite its simplicity, the present device is designed to be highly efficient in removing soft tissue or materials, for example, gelatinous tissue material (such as within an intervertebral discs). Because there is no external suction source or supplemental aspiration required of the present invention to pull material into the cannula, it can further be appreciated that the apparatus is smaller, safer and requires less monitoring than devices that include a separate or external source of suction or additional idler shafts for removing material.

It is also to be appreciated that the apparatus of the present invention may be modified to include a connector for enabling the handpiece to be connected to an external aspiration source. In this case, means for monitoring the vacuum level in the cannula is preferably provided in order to indicate and prevent buildup of excess vacuum in the event the cannula becomes clogged for example. Preferably however, the apparatus is self-contained and requires no external wiring, conduits, tubing or the like.

The present invention further provides a method for treating an intervertebral disc, comprising measuring an intrinsic pressure within an intervertebral disc nucleus and treating the disc based at least in part in the measured pressure within the disc. The step of measuring the pressure is preferably performed before and/or during the step of treating. In addition, the step of measuring comprises monitoring the pressure in a substantially continuous basis over a selected period of time.

In some cases, it may be desirable to introduce material into the disc, for example, artificial nucleus material. The present methods can be easily adapted for such situations.

Accordingly, in one embodiment of the present invention, a method for treating an intervertebral disc comprises the steps of percutaneously removing material, for example nucleus pulposus, from an intervertebral disc through a cannula, and introducing, preferably subsequently introducing a material, for example, a disc replacement material through the same cannula.

The material introduced into the disc may comprise for example, an artificial disc replacement material known in the art. The disc replacement material may comprise a hydrogel, foam, other compressible material, an expandable inflatable element such as a balloon, other suitable disc replacement material, and the like and combinations thereof.

It is to be appreciated that the step of removing material through a cannula is not limited to removing material by means of the apparatus elsewhere described and shown herein but may encompass any suitable means of percutaneously removing the material through a cannula which is then utilized as a passageway for introducing another material, such as an artificial disc replacement material into the disc.

The following specific examples are for purposes of example only, and are not to be considered as limiting the scope of the present invention. As will be appreciated by those of skill in the art, other arrangements and sequencing of steps are possible and are considered to be within the spirit and the scope of the present invention.

### EXAMPLES

Turning now to FIG. 7, in accordance with the present invention, a commercially available pressure transducer 100 is used to monitor pressure within a disc 102, particularly a disc nucleus 104 during a disc decompression procedure. A 17 gauge Crawford style needle 105 is inserted into the skin and spine through the contra-lateral side of the intervertebral disc 102 through the disc annulus 102a prior to the procedure. The distal tip 108 of a 4F piezoelectric transducer catheter 106 is passed through the needle 105. Preferably the distal portion of the transducer catheter 106 has an outer diameter of less than about 2 mm, in order to minimize the invasiveness of the procedure.

The catheter 106 may be mounted to a substantially rigid access device, or may be passed through a substantially rigid access device such as needle 105 shown, to provide direct access to the disc nucleus 104. The needle 105 or access device has a working length, preferably a working length of about 8 inches or less.

A proximal end of the transducer catheter 106 is connected to a cable terminating at a controller/monitor 112. The controller/monitor 112, of generally conventional configuration, includes a DC output with a ring tip sleeve differential female phone plug. The controller 112 provides excitation voltage to produce a calibrated DC output which provides a readout allowing conversion of voltage to psi (pounds per square inch).

A disc decompression procedure is begun. A tool for drawing or suctioning disc nuclear material is inserted through the disc annulus 102a and into the disc nucleus 104. The tool is preferably an embodiment of the nucleotomy apparatus 10 in accordance with the present invention described and shown elsewhere herein. It is to be appreciated that the tool may be passed through the same needle holding the transducer catheter or, alternatively, through a percutaneous incision at a different location within the disc 102, such as shown in FIG. 8.

The pressure transducer tip 108 remains near the center of the disc 102 during the decompression procedure. Alternatively, the pressure transducer may be positioned to measure pressure within a protruding portion of the nucleus pulposus adjacent an affected nerve root for example, or may be positioned within or adjacent any other portion of the intervertebral disc where monitoring pressure is desirable.

The disc pressure is monitored at least once before the decompression procedure and subsequently, at selected time intervals, for example, of about 15 seconds apart, during the procedure. In this specific example, pressure in a lumbar disc is reduced from an initial reading of about 8 psi to about 4 psi after about two minutes of decompression treatment.

In a related example, the pressure monitoring is performed on a thoracic disc which is undergoing a decompression procedure. In this example, pressure is observed on a real time basis as material is removed from the disc. The pressure within the disc decreases from an initial reading of 20 psi to a negative pressure or below atmospheric pressure (i.e. vacuum).

For instance, pressure may be monitored both before and after the procedure in order to determine the need for the treatment procedure and/or the effectiveness or lack of effectiveness of the procedure. A pressure measurement may be taken as a diagnostic tool to determine whether excessive pressure in the disc may be a cause of pain experienced by a patient. If disc pressures appear normal, a physician may choose to select a different pain management therapy. It can also be appreciated that the apparatus and specific techniques for performing a method in accordance with the invention can vary from the examples presented herein without departing from the scope of the invention. The pressure measurement transducer may be designed as a piezoelectric transducer or an optically measured diaphragm transducer. Other options are available as well. The device is preferably connected to a monitor that displays real-time pressures to the physician such that the physician can determine how the surgical procedure is proceeding and when to stop.

The methods of the present invention may further include the step of controlling decompression of the disc, for example by responsively increasing or decreasing the cutting/aspiration power of the decompression device, e.g. tissue removal device, for example in response to a pre-selected disc pressure being detected. In a related example, the methods may include the step of controlling the rate at which the disc is decompressed by causing the tissue removal device to be turned off, or reduced or increased in power, in response to a detected variation in pressure.

In summary, it can be appreciated that the present invention facilitates spinal surgical procedures by providing useful, real-time, pertinent information to a physician before, during and/or after a surgical procedure. For example, the method of the present invention enables a physician to make a calculated determination as to whether to continue or terminate a procedure.

In other words, one significant advantage of the present invention over conventional spinal surgical procedures which do not employ pressure monitoring, is that the present invention allows a more informed diagnosis and a more informed determination of treatment options and the potential effectiveness thereof. In addition, by monitoring pressure in a disc which has previously been treated, a determination can be made as to whether the treatment was successful and whether the condition of the disc has remained stable over time or requires additional treatments.

While this invention has been described with respect to various specific examples and embodiments, it is to be understood that the invention is not limited thereto and that it can be variously practiced within the scope of the following claims.

What is claimed is:

1. An apparatus for removing material from an intervertebral disc of a human or an animal, the apparatus comprising:
a handpiece;
a cannula including a proximal end portion structured to be coupled to the handpiece and an open distal tip structured to be placed in a nucleus of an intervertebral disc of a body; and
a rotational element structured to be operatively coupled to a source of rotational energy, the rotational element disposed at least partially in the cannula and being structured to at least assist in drawing material from an intervertebral disc into the cannula.

2. The apparatus of claim 1 wherein the rotational element and the cannula cooperatively engage to form a source of suction effective in drawing material from an intervertebral disc into the cannula in response to rotation of the rotational element.

3. The apparatus of claim 2 which includes no other source of suction or aspiration.

4. The apparatus of claim 1 wherein the rotational element includes a shaft and one or more outwardly extending projections.

5. The apparatus of claim 4 wherein the one or more outwardly extending projections have a substantially helical configuration.

6. The apparatus of claim 1 wherein the rotational element includes a distal portion that extends beyond the open distal tip of the cannula.

7. The apparatus of claim 6 wherein the distal portion of the rotational element includes one or more outwardly extending projections having a substantially helical configuration.

8. The apparatus of claim 6 wherein the rotational element includes a shaft portion that extends beyond the open distal tip of the cannula.

9. The apparatus of claim 1 wherein the rotational element includes a distal portion that extends a distance in a range of about 0.02 inches to about 1 inch beyond the open distal tip of the cannula.

10. The apparatus of claim 1 wherein the cannula has an outer diameter no greater than about 5 mm.

11. The apparatus of claim 1 wherein the cannula has an outer diameter no greater than about 2 mm.

12. The apparatus of claim 1 wherein the open distal tip of the cannula is beveled.

13. The apparatus of claim 1 wherein the open distal tip of the cannula is substantially perpendicular with respect to a longitudinal axis of the cannula.

14. The apparatus of claim 1 further comprising a collection chamber, in communication with the cannula, structured to contain material passed through the cannula.

15. The apparatus of claim 14 wherein the collection chamber is structured to facilitate quantification of the material removed from an intervertebral disc.

16. The apparatus of claim 1 wherein the cannula is structured to be manually deformable.

17. The apparatus of claim 1 which further comprises a motor operatively coupled to the rotational element to provide rotation to the rotational element.

18. An apparatus for removing tissue from an intervertebral disc of a human or an animal, the apparatus comprising:
a handpiece;
a cannula, having an outer diameter of about 5 mm or less, and including a proximal end portion structured to be coupled to the handpiece and an open distal tip structured to be placed in a nucleus of an intervertebral disc of a human or animal; and
a rotational element structured to cooperate with the cannula in drawing intervertebral disc nucleus tissue into the open distal tip in response to rotation of the rotational element, the rotational element including a shaft and having distal portion extending beyond the open distal tip of the cannula, and a proximal end portion structured to be operatively coupled to a source of rotational energy.

19. The apparatus of claim 18 wherein the rotational element and the cannula are sized and positioned to cooperate to form suction effective in drawing intervertebral disc nucleus tissue into the cannula in response to rotation of the rotational element.

20. The apparatus of claim 18 which includes no other source of suction or aspiration.

21. The apparatus of claim 18 wherein the rotational element includes one or more outwardly extending projections.

22. The apparatus of claim 21 wherein the one or more outwardly extending projections have a substantially helical configuration.

23. The apparatus of claim 18 wherein the shaft extends beyond the open distal tip of the cannula.

24. The apparatus of claim 18 wherein the cannula has an outer diameter no greater than about 2 mm.

25. The apparatus of claim 18 wherein the open distal tip of the cannula is beveled or is substantially perpendicular with respect to a longitudinal axis of the cannula.

26. The apparatus of claim 18 further comprising a collection chamber, in communication with the cannula, structured to contain intervertebral disc nucleus tissue passed through the cannula.

27. The apparatus of claim 18 wherein the cannula is manually deformable.

28. The apparatus of claim 18 which further comprises a motor operatively coupled to the rotational element to provide rotation to the rotational element.

29. A method of removing material from an intervertebral disc of a human or an animal, the method comprising the steps of:
placing into a nucleus of an intervertebral disc of a human or an animal a cannula having an open, distal tip and a rotational element disposed at least partially in the cannula; and
rotating the rotational element relative to the cannula, thereby at least assisting in drawing a material from the nucleus of the intervertebral disc into the open distal tip of the cannula.

30. The method of claim 29 which further comprises passing the material from the nucleus through the cannula.

31. The method of claim 29 wherein the placing step includes percutaneously introducing the cannula into the nucleus of the intervertebral disc, and positioning the open distal tip of the cannula in close proximity to the material from the nucleus to be removed.

32. The method of claim 29 wherein the cannula and rotational element are sized and positioned so that the rotating step is effective to draw the material from the nucleus into the open distal tip of the cannula.

33. The method of claim 29 wherein the cannula has an outer diameter no larger than about 2 mm.

34. The method of claim 29 wherein the material from the nucleus is removed without applying additional suction or aspiration to the open distal tip of the cannula.

35. The method of claim 29 wherein the step of rotating is effective in drawing the material from the nucleus into the open distal tip of the cannula as a substantially single continuous piece.

36. The method of claim 29 further comprising at least one of collecting the removed material from the nucleus and observing the removed material from the nucleus.

37. A method of decompressing an intervertebral disc of a human or an animal, the method comprising the steps of:
positioning a cannula having an open distal tip within a nucleus of an intervertebral disc;
inserting, into the cannula, a rotational element; and
rotating the rotational element, thereby drawing tissue from the nucleus into the cannula.

38. The method of claim 37 wherein the step of rotating includes removing tissue from the nucleus to obtain a desired level of decompression of the intervertebral disc.

39. The method of claim 37 wherein the step of rotating includes removing tissue from the nucleus by suction through the open distal tip of the cannula.

40. The method of claim 37 wherein the step of rotating includes removing tissue from the nucleus through the cannula as a single continuous piece of tissue.

41. A method of treating an intervertebral disc of a human or an animal, the method comprising the steps of:
measuring a pressure within an intervertebral disc; and
treating the disc based at least in part on the measured pressure within the disc.

42. The method of claim 41 wherein the step of measuring is performed before and/or during the step of treating.

43. The method of claim 41 wherein the step of measuring comprises monitoring the pressure within the disc on a substantially continuous basis over a period of time.

44. The method of claim 43 wherein the step of monitoring is performed during the step of treating.

45. The method of claim 41 wherein the step of treating comprises modifying the disc.

46. The method of claim 41 wherein the step of treating comprises removing material from a nucleus of the disc.

47. A method for monitoring a condition of an intervertebral disc, the method comprising:
measuring a pressure in an intervertebral disc after the disc has been surgically treated.

48. The method of claim 47 further comprising the step of treating the intervertebral disc.

49. The method of claim 48 wherein the step of treating comprises removing material from the disc nucleus.

50. The method of claim 48 further comprising the step of monitoring a pressure in the intervertebral disc during the step of treating.

51. The method of claim 50 wherein the step of treating is effective to cause the pressure in the intervertebral disc to be a desired pressure level.

52. A method for treating an intervertebral disc of a human or an animal, the method comprising:
percutaneously removing material from an intervertebral disc through a cannula; and
introducing a disc replacement material into the intervertebral disc through said cannula.

53. The method of claim 52 wherein the step of percutaneously removing comprises removing material from a nucleus of the disc.

54. The method of claim 52 wherein the disc replacement material comprises a material selected from the group consisting of hydrogels, foam materials, inflatable elements and mixtures thereof.
